SEQUENCE LISTING (3 sequences)

SEQ ID NO: 1 - protein, 8 aa, Artificial sequence
  synthetic peptide
  MISC_FEATURE (2)..(2): Xaa represents an arbitrary amino acid
  MISC_FEATURE (7)..(8): Xaa residues either both denote Gly or the first denotes Glu and the second denotes Arg or Lys.
WXHPQFXX

SEQ ID NO: 2 - protein, 8 aa, Artificial sequence
  avidin peptide
WSHPQFEK

SEQ ID NO: 3 - protein, 9 aa, Artificial sequence
  avidin peptide
AWRHPQFGG

(19) United States
(12) Patent Application Publication
(10) Pub. No.: US 2020/0030455 A1
(43) Pub. Date: Jan. 30, 2020

(54) MULTISTEP TUMOR TARGETING BY TAGGED ANTIBODY DERIVATIVES AND TAGGED DRUGS

(71) Applicants: ISTITUTO BIOCHIMICO ITALIANO GIOVANNI LORENZINI S.P.A, Aprilia LT (IT); JOACHIM BERTRAM IBA GMBH, Goettingen (DE); ISTITUTI FISIOTERAPICI OSPITALIERI (IFO) - ISTITUTO REGINA ELENA PER LO STUDIO E LA CURA DEI TUMORI, Roma (IT); UNIVERSITY HOSPITAL JENA, Jena (DE)

(72) Inventors: Joachim BERTRAM, Niemetal (DE); Karlheinz FRIEDRICH, Jena (DE); Leonardo SIBILIO, Roma (IT); Fabio CENTOLA, Roma (IT); Patrizio GIACOMINI, Roma (IT)

(21) Appl. No.: 16/480,805

(22) PCT Filed: Jan. 26, 2018
(86) PCT No.: PCT/IB2018/050476

§ 371 Date: Jul. 25, 2019

(30) Foreign Application Priority Data
Jan. 27, 2017 (IT) 102017000009008

(51) Int. Cl. A61K 47/68; A61K 47/64; A61K 31/537; G01N 33/574

(52) U.S. Cl. CPC A61K 47/6855; A61K 47/6803; G01N 33/57415; A61K 31/537; A61K 47/64

(57) ABSTRACT

The present invention primarily relates to a novel, flexible, modular, multi-step, targeted and conditional drug targeting and delivery system. This system bypasses some general limitations of Antibody Drug Conjugates (ADCs) and widens the applicability of these key anticancer agents in precision medicine.

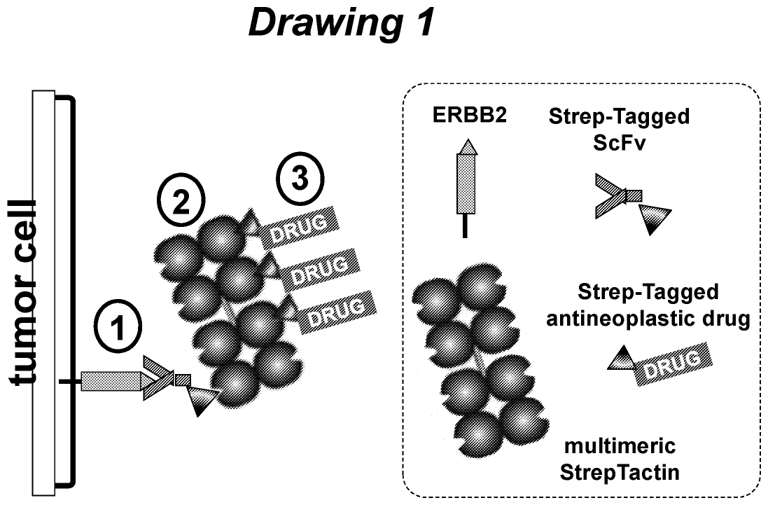

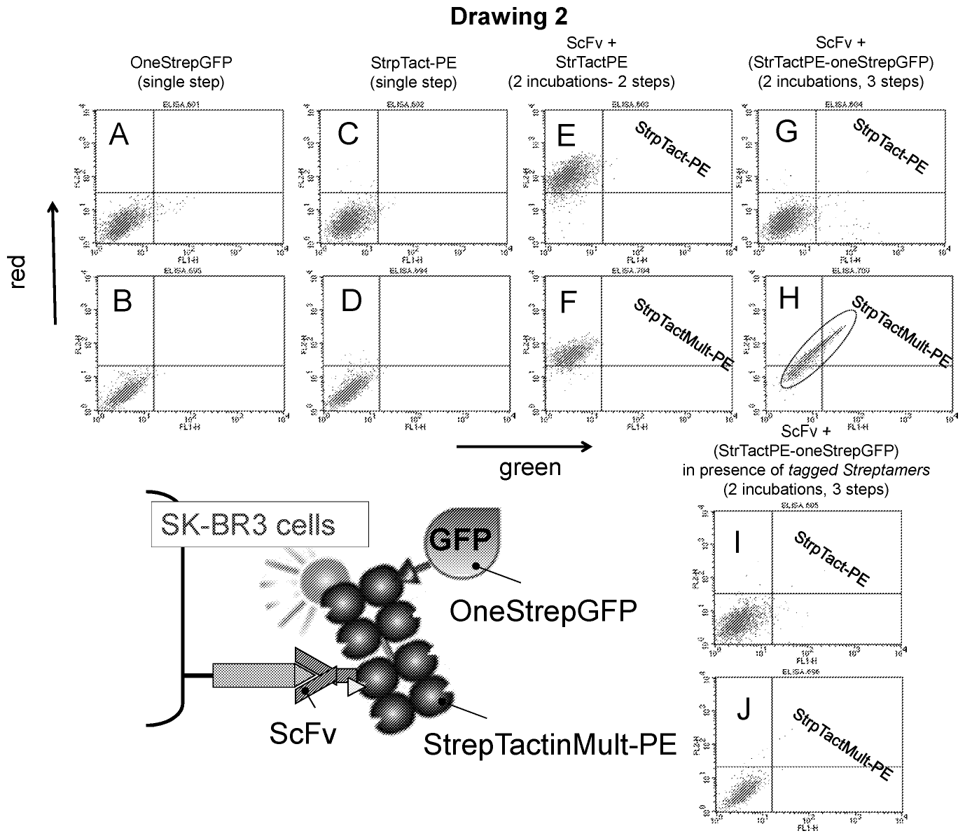

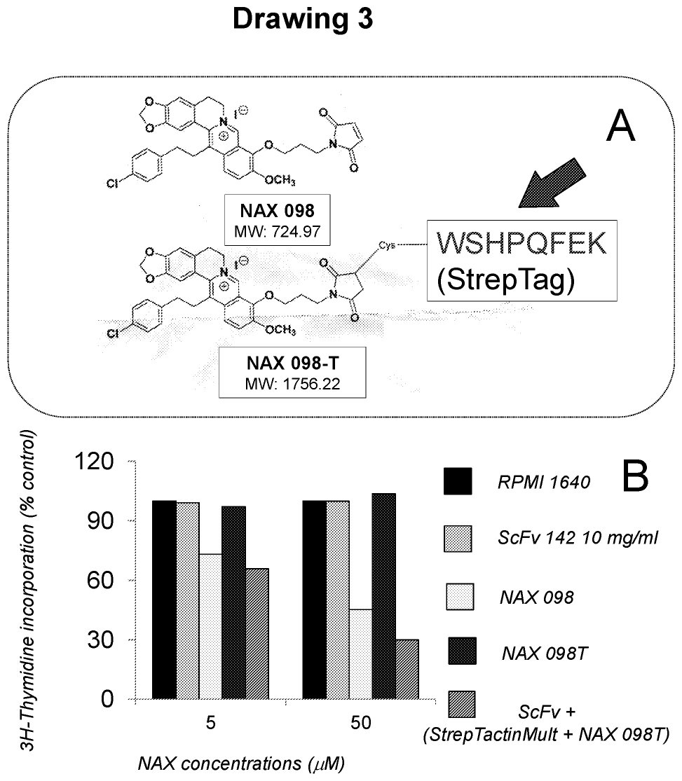

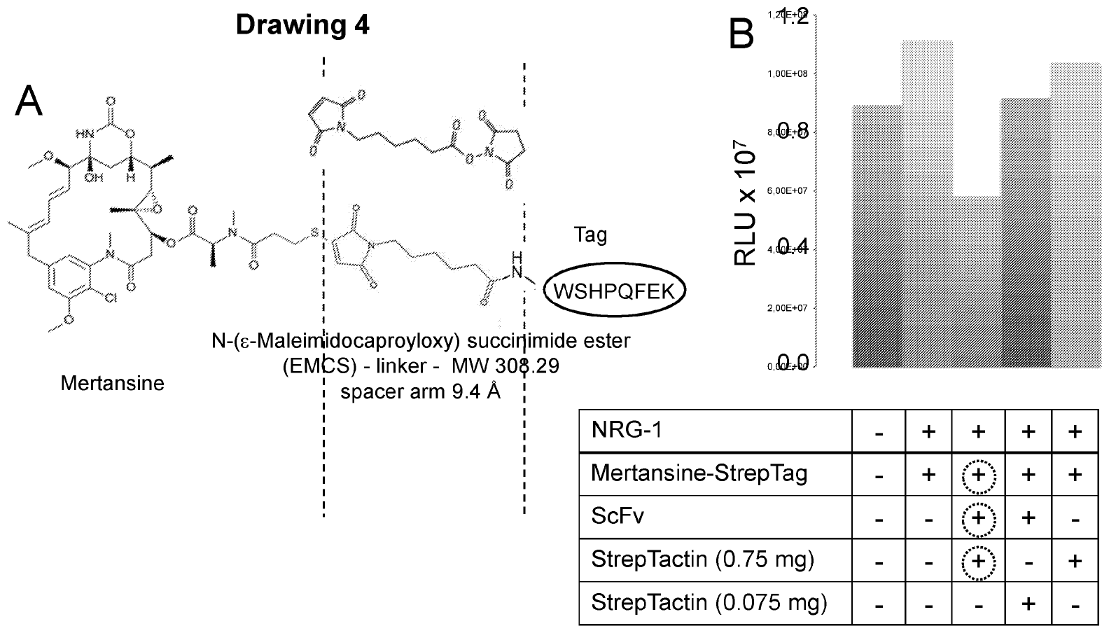

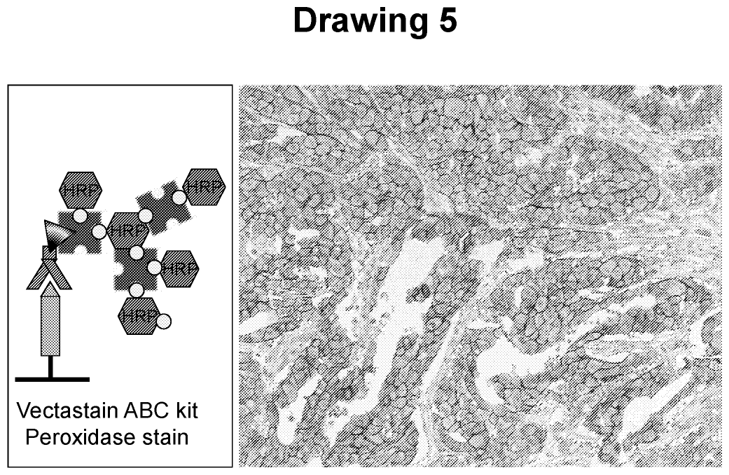

### FIELD OF THE INVENTION

The present invention primarily relates to a novel, flexible, modular, multi-step, targeted and conditional drug targeting and delivery system. This system bypasses some general limitations of Antibody Drug Conjugates (ADCs) and widens the applicability of these key anticancer agents in precision medicine.

### STATE OF THE ART

Cancer results from genetic aberrations such as gene amplifications and mutations. These drive malignant transformation, proliferation, invasion, and metastatic spread (Chaffer C L, Weinberg R A. A perspective on cancer cell metastasis. Science 2011; 331:1559-64. Chaffer C L, Weinberg R A.). Every cancer in every patient displays a unique, dynamic landscape of genetic aberrations, the complexity of which increases during progression, resulting in tumor heterogeneity. The simultaneous presence, in every patient, of many heterogeneous tumor variants is actively selected, acts as a reservoir of escape variants, and represents a major hindrance to therapy (Yap T A, Gerlinger M, Futreal P A, Pusztai L, Swanton C. Intratumor heterogeneity: seeing the wood for the trees. Science translational medicine 2012; 4:127ps10).

Since they target crucial nodes in aberration-driven cancer pathways, antibodies are a preferred therapeutic option. To improve their anticancer properties, and counter secondary resistance, many anti-cancer antibodies have been engineered into Antibody Drug Conjugates (ADCs), e.g. recombinant antibodies that are covalently conjugated with a very active, small antiblastic drug, typically a microtubule inhibitor such as a mayntansinoid (de Goeij B E, Lambert J M. New developments for antibody-drug conjugate-based therapeutic approaches. Curr Opin Immunol 2016; 40:14-23). ADCs combine key advantages of their two constituents: the specific tumor targeting ability of the antibody, and the potent toxic effects of the payload. ADCs such as Trastuzumab-Emtansine (T-DM1) may be used in advanced lines of cancer therapy, particularly after the onset of resistance to their unconjugated counterparts, namely Trastuzumab and Pertuzumab (Verma S, Miles D, Gianni L, et al. Trastuzumab emtansine for HER2-positive advanced breast cancer. N Engl J Med 2012; 367:1783-91). Strikingly, ADCs such as T-DM1 achieve better efficacy and lesser side effects in spite they are administered at lower dosages than their naked antibody counterparts. Their combined administration with conventional antiblastic agents also results in lowering the dosage of the latter, with drastic improvements in toxicity profiles and side-effects (de Goeij B E, Lambert J M. New developments for antibody-drug conjugate-based therapeutic approaches. Curr Opin Immunol 2016; 40:14-23). This is most needed, since standard poly-chemotherapy regimens are almost invariably necessary in advanced cancer.

Unfortunately, ADCs also have limitations, mainly related to their manufacturing pipeline, that requires careful design and development for each ADC, including the optimization of antibody:drug stoichiometry (no more than 6-8 payload molecule per antibody can be tolerated), the careful selection of antibody:drug chemical linkers, and more recently site-specific conjugation strategies to minimize the interference of the drug on the immunoglobulin binding site (de Goeij B E, Lambert J M. New developments for antibody-drug conjugate-based therapeutic approaches. Curr Opin Immunol 2016; 40:14-23). These technical constraints leave little room for ADC potentiation and customization, that are instead most needed to cure a significant fraction of heterogeneous, rapidly evolving cancers in different patients. In principle, medical oncologists may have to face two scenarios: (a) the tumor has developed pharmacological resistance to the drug, but it still reacts with the antibody; (b) the drug is still active, but the tumor has developed into an antigen-loss variant. Both (a) and (b) may be effectively tackled by manufacturing many different ADCs that bear many different payloads. ADCs may then be used in combination and sequentially. However, manufacturing each ADC in as many variants as the number of known active drugs for each tumor type would clearly lead to an exponential expansion of the ADC arsenal in a few years, with inherent hyperbolic costs, and considerable issues and delays when filing documentation and clearing regulatory bodies. To provide a technical solution to these limitations, the inventors have devised a new drug targeting and delivery system. The inventors have combined in a single place several useful instruments necessary to make the ADC technology fully compliant with the biotechnological and clinical challenges of precision medicine.

### SUMMARY OF THE INVENTION

The present invention is primarily based on the surprising observation that the anticancer drugs can be reversibly turned into inactive pro-drugs by the addition of a defined, covalently linked moiety, polypeptidic in nature, here briefly indicated with the name Streptag® and further defined in the description. The Streptag® moiety has two functions: (a) it reversibly inactivates the drug, and (b) it provides ‘handles’ for StrepTactin®, a multivalent adapter, further defined in the description, that non-covalently bridges the tagged drug with a similarly tagged therapeutic anticancer affinity reagent as for example an antibody or fragment thereof. The tagged affinity reagent, the StrepTactin® adapter, and the selected tagged drug bind cancer cells step-wise (typically 3-step) and form a cell surface complex, as depicted in Drawing 1. Thereafter, the tagged pro-drug, that had so far remained in an inactive state to prevent damage to normal cells, becomes reactivated, and exerts targeted antineoplastic effects. This is a novel, flexible, modular, multi-step, targeted and conditional cancer drugging approach that unlike ADCs, can be tailored to combine any antibody specificity with any toxic payload off-the shelf.

Thus, the present invention is a modular anticancer platform with a conserved intermediate module at step 2 (StrepTactin®) allowing the exchange and variable combination of any tagged antibody and StrepTag-inactivated anticancer pro-drug at steps 1 and 3, respectively. The system according to the invention allows tailoring both steps to create a virtually unlimited collection of ADC-like targeting protocols with no need to manufacture many distinct ADCs. Switching and/or combining antibody specificity and/or anticancer agent make it possible to develop families of modular targeting systems suitable for different patients and/or the same patient through disease stages and progression. This effectively addresses some unmet needs of personalised, precision oncology. This is a novel, unanticipated, surprising and ‘universal’ use of state-of-the-art tagging procedures, and an inventive improvement of ADC technology.

Accordingly an object of the invention is a system for drug targeting and delivery comprising:
a mutein of the streptavidin having multiple binding sites for a peptide comprising the amino acid sequence of the formula Trp Xaa His Pro Gln Phe Xaa Xaa (SEQ ID NO:1), in particular wherein said mutein of the streptavidin having a higher binding affinity than wild type-streptavidin for said peptide;
an affinity reagent with a binding activity for a tumour marker linked to a peptide comprising the amino acid sequence of the formula Trp-Xaa-His-Pro-Gln-Phe-Xaa-Xaa (SEQ ID NO: 1);
an anticancer drug linked to a peptide comprising the amino acid sequence of the formula. Trp-Xaa-His-Pro-Gln-Phe-Xaa-Xaa (SEQ ID NO: 1), and wherein in the formula of said sequences Xaa between Trp and His represents an arbitrary amino acid and the two C terminal Xaa residues either both denote Gly or the first denotes Glu and the second denotes Arg or Lys.

### BRIEF DESCRIPTION OF THE DRAWINGS

Drawing 1. A preferred embodiment of the invention and its use is schematically depicted in drawing 1. Tumor cells expressing transmembrane ERBB2 molecules (green) are not targeted directly, as in the case of conventional ADCs, but in a step-wise fashion, as indicated. Step (1): the ScFv (orange) to ErbB2 binds breast cancer cells. This ScFv is tagged with proprietary technology (StrepTag®, yellow triangle). Step (2): proprietary, multimeric, multivalent StrepTactin® (blue) binds cells coated by the tagged ScFv, and spares free tag-binding sites for step 3. Step (3): Strep-tagged anticancer drugs are redirected against ErbB2-expressing tumors.

Drawing 2. High-multiplicity StrepTactin supports the approach of the system according to the present invention. High-multiplicity StrepTactin® redirects a tagged Green Fluorescent Protein (OneStrepGFP) onto cultured human breast cancer cells (SK-BR-3) overexpressing ERBB2.

Drawing 3. The system according to the present invention enables anticancer drug targeting with a berberine derivative named NAX98T. StrepTagging (outlined in panel A) inactivates NAX 098. The system redirects the tagged anticancer drugs toward ERBB2-overexperessing tumor cells, and the drugs are unexpectedly reactivated upon cell contact, as shown by the inhibition of thymidine incorporation into tumor cell DNA.

Drawing 4. The system according to the present invention enables anticancer targeting with tagged Mertansine. StrepTagging of Mertansine (panel A) inactivates the drug that becomes reactivated (as in the above example) upon system-mediated dispatch to tumor cells. The antitumor effect in this case is monitored by a c-fos promoter/Green Fluorescent Protein (GFP) reporter system. The system according to the invention interferes with downstream ERBB2 signaling (c-fos activation) by quenching green light emission, both spontaneous and induced by stimulation with the major ERB ligand Neuregulin-1 (NRG-1).

Drawing 5. The system according to the present invention as a diagnostic tool. The system according to the invention is a general vision applicable to in vitro diagnostics aimed at defining breast cancer molecular classes without subverting the pathological routine. Commonly employed pathology assays based on biotin may be incorporated in the protocol, with no need of substantial modification.

### DETAILED DESCRIPTION OF THE INVENTION

The system for drug targeting and delivery according to the present invention comprises a mutein of streptavidin having multiple binding sites for a peptide ligand comprising the amino acid sequence Trp Xaa His Pro Gln Phe Xaa Xaa (SEQ ID NO: 1) wherein Xaa between Trp and His represents an arbitrary amino acid, and the two C terminal Xaa residues either both denote Gly or the first denotes Glu and the second denotes Arg or Lys.

The term multiple binding sites means that the mutein has at least two distinct binding sites for the peptide ligand. According to one embodiment the mutein comprises 2, 4, 8, 10 or more distinct binding sites for the peptide ligand. Muteins suitable for use in the system of the invention are all streptavidin mutants able to bind at least two different peptide ligands, preferably with a higher binding affinity than wild type-streptavidin.

Streptavidin muteins suitable to be used in the system of the invention are disclosed in U.S. Pat. No. 6,103,493 (herein incorporated by reference). For example, Streptavidin muteins contain at least one mutation in the region of the amino acid positions 44 to 53 with reference to the amino acid sequence of wild type streptavidin as set forth for example in the protein databank Uniprot, e.g. entry name P22629 (SAV_STRAV). Preferably, at least one mutation is present in the region of amino acid positions 44 to 47, more preferably wherein Glu is replaced by a hydrophobic aliphatic amino acid at position 44, an arbitrary amino acid is present at position 45, a hydrophobic aliphatic amino acid is present at position 46 or/and Val is replaced by a basic amino acid at position 47, more preferably Val-Thr-Ala-Arg is present in the region of amino acid positions 44 to 47.

According to one embodiment the mutein is the protein marketed by IBA with the name Streptactin®. According to a preferred embodiment, the multimeric mutein streptavidin is a high-multiplicity StrepTactin that incorporates at least 8 distinct StrepTactin, preferably between 8-10 subunits. Mutein streptavidin may be for example a homo dimer, homo tetramer, homo-heptamer, etc, made up of identical subunits.

This high-multiplicity multimeric mutein streptavidin has been modified to meet pre-defined specifications, e.g. a greater ability to multimerize and crosslink distinct chemical species, as described below. These higher-order multimers allow for a higher multiplicity of interactions of StrepTactin with tagged proteins or other chemical entities such as small drugs.

The system for drug targeting and delivery according to the present invention further comprises an affinity reagent with a binding activity for a tumour marker. Such affinity reagent is linked to a peptide comprising the amino acid sequence Trp Xaa His Pro Gln Phe Xaa Xaa (SEQ ID NO: 1), wherein Xaa between Trp and His represents an arbitrary amino acid and the two C terminal Xaa residues either both denote Gly or the first denotes Glu and the second denotes Arg or Lys.

The affinity reagents for use in the invention bind specifically tumour markers. According to one preferred embodiment the tumour marker is the Receptor tyrosine-protein kinase ErbB-2, also known as ERBB2, Neu, Her2, and CD340.

The affinity reagent may be any protein with a specific affinity to a tumour marker, for example an antibody, a whole antibody, or a functional fragment thereof or an antibody mimetic, preferably monoclonal antibodies. The affinity reagent may be a chimeric antibody, a human antibody, a humanized antibody, a single chain antibody (scFV), a defucosylated antibody or a bispecific antibody. Functional antibody fragments include a UniBody, a domain antibody or a Nanobody. Antibody mimetics include an Affibody, a DARPin, an Anticalin, an Avimer, a Versabody or a Duocalin.

According to one embodiment, the affinity reagent is an antibody, or a functional fragment thereof that specifically binds ErbB-2, for example Pertuzumab, Trastuzumab or the scFv disclosed in IT Patent Application n:102016000033776. Other examples of antibodies that may be effectively tagged and incorporated in the system of the invention are bevacizumab, cetuximab, alemtuzumab, trastuzumab, pertuzumab, rituximab and the like, in particular trastuzumab and pertuzumab.

The system for drug targeting and delivery according to the present invention further comprises an anticancer drug linked to a strep-tag peptide of SEQ ID:1.

The anticancer drugs include, but are by no means limited to, DNA nanobinders such as Berberine and its derivatives, as well as StrepTagged inhibitors of microtubule assembly widely used to prepare ADCs, such as Mertansine. According to one embodiment of the invention, the term drug identifies certain derivatives of berberin, more specifically the compound 9-(3-propylmaleim ide)-13-[2-(4-chlorophenyl)ethyl]berberine, named NAX 098T, as well as the antiblastic agents used to manufacture ADCs, namely the thiol-containing mayntainsinoid derivatives, more specifically Mertansine/DM-1. However, it will be apparent to those skilled in the art that many classes of compounds can be similarly tagged and manipulated to incorporate them in the system according to the invention.

Preferably, the antineoplastic agent is selected from the group consisting of small molecules, nanoparticles, antimetabolites, alkylating agents, topoisomerase inhibitors, microtubule-targeting agents, kinase inhibitors, protein synthesis inhibitors, immuno-therapeutics, hormones or analogs thereof, DNA nanobinders, and/or mTOR inhibitors.

Other specific anticancer drugs to be used in the system as described and claimed herein include for example, chemotherapeutic agents such as Paclitaxel, Anthracyclines, Fluoropirimidine, vinca alkaloids, platinum salts, in particular capecitabine, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, melphalan, cyclophosphamide, 6-mercaptopurine, 6-thioguanine, cytarabine (CA), 5-azacytidine, hydroxyurea, deoxycoformycin, 4-hydroxyperoxycyclophosphor-amide, 5-fluorouracil (5-FU), 5-fluorodeoxyuridine (5-FUdR), methotrexate (MTX), colchicine, taxol, vincristine, vinblastine, etoposide, trimetrexate, teniposide, cisplatin and diethylstilbestrol (DES).

As disclosed above, the anticancer drug and the affinity reagents are linked to a peptide comprising the amino acid sequence with the formula Trp Xaa His Pro Gln Phe Xaa Xaa (SEQ ID NO: 1) wherein Xaa between Trp and His represents an arbitrary amino acid and the two C terminal Xaa residues either both denote Gly or the first denotes Glu and the second denotes Arg or Lys. Peptides suitable to be used in the system of the invention are disclosed in U.S. Pat. No. 5,506,4121 (herein incorporated by reference). According to one embodiment, the anticancer drug is linked to a peptide with the sequence WSHPQFEK (SEQ ID NO:2) or AWRHPQFGG (SEQ ID NO:3). According to one embodiment, the peptide is the peptide Strep-tag® or Strep-tag II commercially marketed by IBA. These peptides bind StrepTactin with much higher affinity than biotin. Therefore, it will be apparent to those skilled in the art that the StrepTactin:StrepTag system minimizes possible in vivo interference by natural biotin present in competing amounts in the circulation and body fluids. Moreover, it is also apparent that coupling therapeutic proteins with the StrepTag does not need being conducted chemically, since the Tag technology embedded in the invention permits to incorporate appropriate numbers of tags in the primary polypeptide sequence, with no need to modify these molecules post-synthesis. These post-synthesis manipulations would instead be necessary to tag proteins with biotin. Post-synthesis modifications of different kinds are also necessary to manufacture ADCs.

Drawing 1 depicts the use of a preferred embodiment of the system according to the present invention wherein a Single chain Fragment of variable antibody region (ScFv) directed to ERBB2 (namely W6/800, patented by IBI Lorenzini, Aprilia, Italy and disclosed in IT Patent Application n:102016000033776.) is tagged by the short polypeptide (WSHPQFEK) SEQ ID NO 2 patented by IBA Goettingen, Germany and disclosed in U.S. Pat. No. 5,506,121. The tagged ScFv binds breast cancer cells (see step 1) and directs a tag-specific, multivalent protein adapter (a special formulation of the recombinant protein StrepTactin patented by IBA Goettingen and disclosed in U.S. Pat. No. 6,103,493) toward tumor cells (step 2). The adapter is engineered to display extremely high avidity and affinity for the tag of the binder. In addition, upon interaction with the binder on the cell surface, the adapter conserves free tag-binding sites. These free valences enable redirection toward cancer cells of suitably StrepTagged anticancer drugs (step 3).

A further aspect of the present invention relates to a pharmaceutical composition comprising the reagents of the system according to the present invention, for example the reagents used in steps 1-3 of drawing 1. The term “composition” as employed herein comprises at least one compound of each kind in optimized relative quantities. Preferably, such a composition is a therapeutical/pharmaceutical or a diagnostic composition. The invention also provides a pharmaceutical pack or kit comprising one or more containers filled with one or more of the ingredients of the system of the invention.

The composition preferably comprises a pharmaceutically acceptable carrier, diluent and/or excipient. In a specific embodiment, the term “pharmaceutically acceptable” means suitable for approval by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, for example in purified form, together with a suitable amount of carrier so as to provide the form for proper administration to the subject. The formulation should suit the mode of administration.

In one embodiment, for example, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example as a dry lyophilized powder or water-free concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention may be administered locally (e.g. for the loco-regional therapy of endocavitary neoplastic exudates) or systemically. According to an especially preferred embodiment the composition comprises a further anticancer drug or chemical antineoplastic agent. Preferably, the composition of the invention is used in combination with at least one further antineoplastic agent. Said combination is effective, for example, in inhibiting abnormal cell growth. Many antineoplastic agents are presently known in the art. In general, the term includes all agents that are capable of prevention, alleviation and/or treatment of hyperproliferative disorders. Especially preferred are antineoplastic agents such as inducers of apoptosis, DNA nanobinders, inhibitors of aromatase and other antineoplastic agents.

Preferably the antineoplastic agent is selected from the group consisting of small molecules, nanoparticles, antimetabolites, alkylating agents, topoisomerase inhibitors, microtubule-targeting agents, kinase inhibitors, protein synthesis inhibitors, immuno-therapeutics, hormones or analogs thereof, DNA nanobinders, and/or mTOR inhibitors.

The compositions of the invention may be administered in combination with a further therapeutic composition comprising an active agent as described above and/or irradiation and/or radiotherapy. According to a preferred embodiment, the system and the compositions of the invention are for the use in treating and/or preventing and/or diagnosing proliferative disorders, in particular neoplastic diseases and cancer. The hyperproliferative disease is preferably selected from disorders associated with, accompanied by, or caused by ERBB2 expression, overexpression or hyperactivity, such as cancer, in particular breast, ovarian, prostate and lung cancer, but also gastric carcinoma, glioblastoma, uterine carcinosarcoma, fallopian tube cancer, endometrial carcinoma, and bladder transitional cell carcinoma. In particular, for these tumors, it has been demonstrated a role of ERBB2 in promoting cancer development and growth. Thus, the inhibition of this protein could be beneficial. Antibodies and step 1 reagents with different specificities may have distinct spectra of activity toward other neoplastic diseases.

The amount of the compounds of the invention which will be effective in the treatment of cancer can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 1-20 mg (therapeutic antibodies), and 1-10 mg (ADCs) per kilogram body weight, each antibody molecule containing from 2 to 8 molecules of active compounds. Effective doses of the present drug targeting system may be extrapolated from dose-response curves derived from in vitro or animal model test systems.

The composition may comprise a further active agent, the active agent may be a therapeutic antibody or antibody fragment or an anti-neoplastic agent, preferably selected from the group of small molecules, antimetabolites, alkylating agents, topoisomerase inhibitors, microtubule-targeting agents, kinase inhibitors, protein synthesis inhibitors, immuno-therapeutics, hormones or analogs thereof, and/or mTOR inhibitors. The compositions may be administered for example intravenously, intramuscularly, and/or subcutaneously and in combination with a further therapeutic composition and/or irradiation.

Yet another aspect of the present invention is a method for diagnosing (in vitro, in vivo or ex vivo) a cancer associated with ERBB2 in a subject, comprising the following steps:
(a) contacting a sample of cells obtained from said subject with:
   i) an affinity reagent, in particular an antibody or antigen binding portion thereof, having a specific binding activity to ErbB2, wherein said affinity reagent is linked to a peptide comprising the amino acid sequence of the formula Trp Xaa His Pro Gln Phe Xaa Xaa (SEQ ID NO:1);
   ii) a mutein of the streptavidin having a higher binding affinity than wild type-streptavidin for a peptide comprising the amino acid sequence of the formula Trp Xaa His Pro Gln Phe Xaa Xaa (SEQ ID NO:1) with at least two binding sites for said peptides;
   iii) a label for diagnostic use linked to a peptide comprising the amino acid sequence of the formula. Trp-Xaa-His-Pro-Gln-Phe-Xaa-Xaa (SEQ ID NO: 1),
   and wherein in said sequences Xaa between Trp and His represents an arbitrary amino acid and the two C terminal Xaa residues either both denote Gly or the first denotes Glu and the second denotes Arg or Lys.
(b) measuring the level of binding to ErbB2 on the cells, wherein abnormally high levels of binding to ErbB2 indicate that the subject has a cancer associated with ErbB2.

In the context of the present invention, “abnormally high” means higher binding levels of ErbB2 compared to a healthy subject having no cancer. Preferably the subject is an animal, more preferably a mammalian and in particular a human. It will be apparent to those skilled in the art that the StrepTag in step 3 can be further coupled to other moieties for, e.g., imaging applications. Thus, for diagnostic purposes, the ScFv of the invention may redirect labels. Suitable step 3 labels include radioactive labels, fluorescent labels, dye groups, enzyme labels, chromogenes, chemiluminescent groups, biotinyl groups, predetermined polypeptide epitopes recognized by a secondary reporter etc. These may be used in immunohistochemistry assays or for molecular imaging in vivo (immunoscintigraphy) and ex vivo (intra-operatory).

### EXAMPLES

Materials and Methods:

SK-BR-3 human breast cancer cells (5×10⁵) overexpressing ERBB2 were incubated on ice (successive incubations of 30 minutes each) in 50 μl Phosphate (0.01M) buffered (pH 7.4) saline (0.9%), i.e. PBS, containing one or more of the following reagents, as indicated: (i) tagged ScFv (0.5 μg); (ii) standard Phycoerithrin-conjugated StrepTactin (StrptTact-PE) multimerized at an average theoretical multiplicity of 6 (0.75 μg); (iii) high multiplicity Phycoerithrin-conjugated StrepTactin (StrptTactMult-PE) multimerized at an average theoretical multiplicity of 10 (0.75 μg); (iv) StrepTagged Green Fluorescent Protein (OneStrepGFP; 0.75 μg). At the end of each incubation the cells were washed twice with PBS. At the end of the last incubation, the cells were washed twice and analyzed by two-color flow cytometry in a FACScan (B&D).

Results:

Panels A-D (Drawing 2) show that the 3 fluorescent reagents (2 distinct StrepTactin-PE prepared at two different multiplicities, and a single preparation of OneStrepGFP) display no detectable background stain when individually incubated with ERBB2-expressing breast carcinoma cells, and read in the red and green channels, respectively. Panels E and G (Drawing 2) depict experiments in presence of conventional, commercially available (IBA Goettingen, see patent n. DE 196.41.876.10, Strep-Tactin: “Streptavidin muteins”) StrptTact-PE, whereas panels F and H depict experiments in presence of tailored, high multiplicity StrptTactMult-PE. Comparison of panels E and F shows that the tagged ScFv drives both PE-labelled StrepTactin preparations onto the cells with comparable efficiency (see fluorescence reading in the red channel). Comparison of panels G and H demonstrates that the tagged ScFv can simultaneously drive onto the cells StrepTactin-PE and OneStrepGFP, but only when StrepTactinMult-PE is employed (double-color fluorescence in H, circled), whereas conventional low-multiplicity StrepTactin-PE fails to capture OneStrepGFP, and in addition it is displaced from the cell surface by OneStrepGFP addition (G compared to E). This is very surprising and unexpected, since the valences theoretically available on conventional StrepTactin were expected to suffice for the generation of an appropriate multimerization lattice. In agreement with a critical lack of valences, OneStrepGFP binding to StrepTactinMult-PE was similarly competed and abrogated by other StrepTagged proteins of irrelevant specificity (compare H and J). Thus, using OneStrepGFP as a readout, a novel StrepTactin variant was developed, and a precise stoichiometry of the reagents used in the three targeting steps was identified. A graphical representation of the experiment is depicted in the cartoon.

The system according to the invention enables anticancer drug targeting by NAX 098T. Depicted in drawing 3 and described below.

Materials and Methods:

A modified DNA nanobinder (a berberin derivative named NAX 098, depicted in A) was synthesized and selected among other similar compounds. NAX 098 was modified by covalent addition of the polypeptide StrepTag (WSHPQFEK), as also shown in (A). SK-BR-3 breast carcinoma cells were seeded in 96-well plates (quadruplicates), and cultured for 30 min in the presence of ScFv (10 μg/ml). Meanwhile, equal molar concentrations of NAX 098 and NAX 098T (or RPMI 1640 medium, as a control) were pre-incubated with StrepTactMult in complete culture medium (RPMI 1640 containing 10% bovine serum) to form drug: StrepTactin complexes. The drug: StrepTactin complexes were added to separate cultures to yield a final StrpTactMult concentration of 0.75 μg/well (approximately 3.2 μg/ml), and two final drug concentrations of 5 and 50 μM, as indicated in (B). As a control, equal amounts of NAX 098 and NAX 098T drugs were added to separate wells in the absence of the Toolbox cocktail. All the cells were cultured for 72 h, and ³H-Thymidine incorporation was assessed over a 4 h radioactive pulse. Results were expressed as percent of radioactive Thymidine incorporation as compared to untreated (RPMI 1640 vehicle alone) control cells.

Results:

As expected, NAX 098 strongly inhibited cell proliferation (yellow bars). In contrast, and very surprisingly, NAX 098T was ineffective (blue histograms), demonstrating that StrepTagging abrogates the biological activity of the anticancer drug. However, incubation of breast carcinoma cells with ScFv in the first step and the NAX 098T: StrepTactMult complexes in the second step fully restored the antiproliferative effect (orange bars). Thus, StrepTagging unexpectedly detoxifies the drug, and the tagged ScFv not only retargets the drugs onto the tumor, but also makes tumor drugging more specific, conditional, and completely ScFv-dependent.

### Preparation of StrepTagged NAX098

### Synthesis of 13-[2-(4-chlorophenyl)ethyl]berberrubine

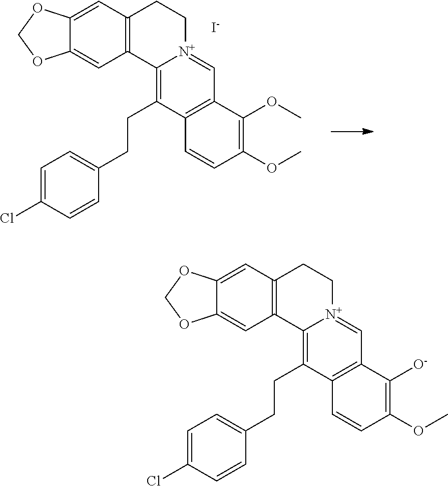 [SMILES: COc1ccc2c(CCc3ccc(Cl)cc3)c3[n+](cc2c1OC)CCc1cc2c(cc1-3)OCO2 ; [I-] ; COc1ccc2c(CCc3ccc(Cl)cc3)c3[n+](cc2c1[O-])CCc1cc2c(cc1-3)OCO2]

13-[2-(4-chlorophenyl)ethyl]berberine (BioFactors, 2013, 39, 672) (1.138 g, 1.88 mmol) was dissolved in dry DMF (15 mL) and stirred at 150° C. during 6 hours. Evaporation of the solvent at reduced pressure gave a crude material which was precipitated with methyl t-butyl ether, filtered and purified by flash chromatography on silica gel (CH₂Cl₂-MeOH, 88:12) to yield the title compound (88%).

¹H NMR (400 MHz, CDCl3), δ (ppm)=3.7 (s, 2H); 3.0 (s, 2H); 3.45 (m, 2H); 3.95 (s, 3H); 4.3 (m, 2H); 6.1 (m, 2H); 6.75 (m, 2H); 6.85 (m, 2H); 6.9 (m, 2H); 7.15 (m, 3H); 7.35 (m, 1H); 9.45 (s, 1H).

### Synthesis of 9-(3-propylmaleimide)-13-[2-(4-chlorophenyl)ethyl]berberine (NAX098)

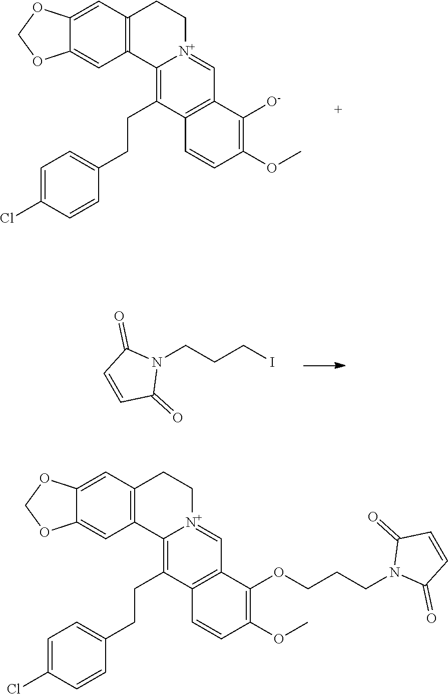 [SMILES: COc1ccc2c(CCc3ccc(Cl)cc3)c3[n+](cc2c1[O-])CCc1cc2c(cc1-3)OCO2 ; O=C1C=CC(=O)N1CCCI ; COc1ccc2c(CCc3ccc(Cl)cc3)c3[n+](cc2c1OCCCN1C(=O)C=CC1=O)CCc1cc2c(cc1-3)OCO2]

13-[2-(4-chlorophenyl)ethyl]berberrubine (65 mg, 0.141 mmol) and 1-(3-iodopropyl)-1H-pyrrole-2,5-dione (38 mg, 0.141 mmol, Chembiochem, 2008, 9, 552-64) were dissolved in dry DMF (2 mL) and stirred at 100° C. during 5 hours. Evaporation of the solvent at reduced pressure gave a crude material which was purified by flash chromatography on silica gel (CH₂Cl₂-MeOH, 96:4) to yield the title compound (43%).

¹H NMR (400 MHz, CD3OD), δ (ppm)=1.18 (s, 3H); 2.23-2.11 (m, 4H); 2.87-2.78 (m, 4H); 3.00 (t, 4H); 3.20 (s, 1H); 3.35-3.28 (m, 24H); 3.85 (t, 4H); 3.92 (t, 4H); 4.12 (s, 6H); 4.41 (t, 4H); 4.76 (s, 3H); 4.86 (s, 26H); 6.12 (s, 4H); 6.74 (d, 4H); 6.84 (s, 4H); 6.91 (s, 2H); 7.06 (d, 4H); 7.31 (s, 2H); 8.18 (d, 2H); 8.30 (d 2H); 9.91 (s, 2H).

Coupling of NAX098 with Strep Tag

Using the maleimide group, the compound NAX098 was linked to the streptag pepetide WSHPQFEK (SEQ ID NO 2).

The peptide was automatically synthesized from an Autospot robot (Intavis, Cologne) according to standard procedure. Following the peptide was cleaved from the resin and purified by HPLC using water and acetonitrile as eluent in gradient+0.1% TFA. The peptide was linked to NAX098 coupling it in an equimolar ratio at room temperature in DMF for 4 hours. The DMF was evaporated under vacuum and the mixture was purified by HPLC.

The coupled product was analyzed by MALDI-MS (m/z) 1819.89 (M-I-) NAX098 with strep-tag II.

The system according to the invention enables anticancer drug targeting by StrepTagged Mertansine. Depicted in drawing 4 and described below.

Materials and Methods:

Mertansine was StrepTagged using a linker-spacer (depicted in A). The Strep-tag peptide WSHPQFEK (SEQ ID NO 2) was linked to the mertansine thiol group using the protocol disclosed in Bioconjugate Techniques, 3rd Edition Bioconjugate Techniques, 3rd Edition (2013) by Greg T. Hermanson. BRC 230 breast carcinoma cells, expressing moderate levels of ERBB2 were stably transfected with a promoter-reporter gene construct in which luciferase is placed under the control of the c-Fos promoter. Oncogenic intracellular signalling cascades, most notably the Ras-Raf-MAPK-ERK and PI3K-AKT pathways, involved in cell proliferation and cell survival, respectively, converge on cFos (5). Therefore, c-Fos activation is proportional to ERBB2 signaling, and luminescence is expected to decrease in response to the Toolbox treatment. BRC transfectants were seeded in 96-well plates (quadruplicates), and cultured for 24 h either in the presence or in the absence of the ERB-specific growth factor Neuregulin 1 (NRG-1) at a concentration of 5 nM, as indicated. During the last 30 min of this incubation ScFv (10 μg/ml) was added, where indicated. Meanwhile, equal molar concentrations of StrepTagged Mertansine (or RPMI 1640 medium, as a control) were pre-incubated with StrepTactMult in complete culture medium (RPMI 1640 containing 10% bovine serum) to form drug: StrepTactin complexes. The drug: StrepTactin complexes were added to separate cultures to yield a final StrpTactMult concentration of 0.75 μg/well (approximately 3.2 μg/ml), and a final drug concentrations of 5 nM, e.g. approximately three orders of magnitude less than NAX 098T. This experiment is depicted in (B). All the cells were cultured for additional 72 h, and Relative Luminescence Units (RLU) were assessed in a luminometer.

Results:

B: as expected (5), NRG-1 treatment enhanced the activity of the ERBB2 pathway (compare blue and green bars). Toolbox treatment in the presence of a StrepTagged Mertansine inhibited the ERBB2 pathway, and the inhibition was proportional to the amounts of the StrepTactin adapter present in the mix (orange and red bars). StrepTactin alone had a negligible effect (purple bars), demonstrating that Toolbox redirects StrepTagged Mertansine onto breast carcinoma cells.

The system according to the invention as a diagnostic tool. Depicted in drawing 5 and described below.

Materials and Methods:

A cryostatic section from an ERBB2-overexpressing breast cancer lesion was stained by the ScFv and washed in Phosphate (0.01 M) buffered (pH 7.0) Saline (0.9%). Binding was revealed by a commercially available avidin-biotin amplification system (Vectastain, Vector Laboratories) exactly as recommended by the manufacturer. The system includes multivalent HorseRadish Peroxidase (HRP) complexes, and StrepTactin binds both the StrepTag and biotin moieties on HRP.

Results:

Strong specific staining of the tumor lesion is seen with sharp boundaries separating ErbB2-overexpressing cells (malignant) from normal stroma (not stained). Showing that StrepTagged ScFv can be recognized by standard immunodiagnostic reagents.

LEGEND OF THE SEQUENCES

| | |
|---|---|
| SEQ ID NO 1 | WXHPQFXX (Trp Xaa His Pro Gln Phe |
| | Xaa Xaa); |
| SEQ ID NO 2 | WSHPQFEK; |
| SEQ ID NO: 3 | AWRHPQFGG. |

1. A system for drug targeting and delivery comprising:
a mutein of the streptavidin having multiple binding sites for a peptide comprising the amino acid sequence of the formula Trp Xaa His Pro Gln Phe Xaa Xaa (SEQ ID NO: 1);
an affinity reagent with a binding activity for a tumour marker linked to a peptide comprising the amino acid sequence of the formula Trp-Xaa-His-Pro-Gln-Phe-Xaa-Xaa (SEQ ID NO: 1);
an anticancer drug linked to a peptide comprising the amino acid sequence of the formula. Trp-Xaa-His-Pro-Gln-Phe-Xaa-Xaa (SEQ ID NO: 1), and wherein in said sequences Xaa between Trp and His represents an arbitrary amino acid and the two C terminal Xaa residues either both denote Gly or the first denotes Glu and the second denotes Arg or Lys.

2. The system according to claim 1 wherein said affinity reagent is selected from an antibody, a whole antibody, or a functional fragment thereof or an antibody mimetic, a monoclonal antibodies, a chimeric antibody, a human antibody, a humanized antibody, a single chain antibody (scFV), a defucosylated antibody, a bispecific antibody, a UniBody, a domain antibody or a Nanobody.

3. The system according to claim 1 wherein said tumour marker is the Receptor Tyrosine protein Kinase ErbB-2.

4. The system according to claim 1 wherein said anticancer drug is linked to a peptide comprising the amino acid sequence WSHPQFEK (SEQ ID NO:2) or AWRHPQFGG (SEQ ID NO:3).

5. The system according to claim 1 wherein said anticancer drug is selected from Mertansine or other inhibitors of microtubule assembly widely used to prepare ADCs, berberin or its derivatives, 9-(3-propylmaleimide)-13-[2-(4-chlorophenyl)ethyl]berberine (named NAX 098T), thiol-containing mayntainsinoid derivatives, DNA nanobinders, and/or mTOR inhibitors, chemotherapeutic agents such as Paclitaxel, Anthracychnes, Fluoropirimidine, vinca alkaloids, platinum salts, in particular capecitabine, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, melphalan, cyclophosphamide, 6-mercaptopurine, 6-thioguanine, cytarabine (CA), 5-azacytidine, hydroxyurea, deoxycoformycin, 4-hydroxyperoxycyclophosphor-amide, 5-fluorouracil (5-FU), 5-fluorodeoxyuridine (5-FUdR), methotrexate (MTX), colchicine, taxol, vincristine, vinblastine, etoposide, trimetrexate, teniposide, cisplatin and diethylstilbestrol (DES).

6. A pharmaceutical composition or kit comprising the system according to claim 1 and a pharmaceutically acceptable carrier, diluent and/or excipient.

7. A method of treating a hyperproliferative diseases, in particular a neoplastic disease or cancer comprising administering to a subject in need thereof a therapeutically effective dose of the composition according to claim 6.

8. The method of claim 7, further comprising administering to the subject an antineoplastic agent.

9. The method of claim 7 wherein the hyperproliferative disease is associated with, accompanied by, or caused by ErbB2 expression, overexpression or hyperactivity.

10. The method of claim 7 wherein said hyperproliferative disease is a neoplastic disease or a cancer selected from a breast cancer, ovarian cancer, prostate cancer, lung cancer, gastric carcinoma, glioblastoma, uterine carcinosarcoma, fallopian tube cancer, endometrial carcinoma or bladder transitional cell carcinoma.

11. An in vitro method for diagnosing a cancer associated with ErbB2 in a subject, comprising the following steps:
(a) contacting a sample of cells obtained from said subject with:
i) an affinity reagent, in particular an antibody or antigen binding portion thereof, having a specific binding activity to ErbB2, wherein said affinity reagent is linked to a peptide comprising the amino acid sequence of the formula Trp Xaa His Pro Gln Phe Xaa Xaa (SEQ ID NO: 1);
ii) a mutein of the streptavidin having a higher binding affinity than wild type-streptavidin for peptide comprising the amino acid sequence of the formula Trp Xaa His Pro Gln Phe Xaa Xaa (SEQ ID NO: 1) with at least two binding sites for said peptides;
iii) a label for diagnostic use linked to a peptide comprising the amino acid sequence of the formula. Trp-Xaa-His-Pro-Gln-Phe-Xaa-Xaa (SEQ ID NO: 1), and wherein in said sequences Xaa between Trp and His represents an arbitrary amino acid and the two C terminal Xaa residues either both denote Gly or the first denotes Glu and the second denotes Arg or Lys.
(b) measuring the level of binding to ErbB2 on the cells, wherein abnormally high levels of binding to ErbB2 indicate that the subject has a cancer associated with ErbB2.

12. The method according to claim 11 wherein the subject is a human and wherein said label is selected from radioactive labels, fluorescent labels, dye groups, enzyme labels, chromogenes, chemiluminescent groups, biotinyl groups, predetermined polypeptide epitopes recognized by a secondary reporter.

13. An anticancer drug linked to a peptide comprising the amino acid sequence of the formula. Trp-Xaa-His-Pro-Gln-Phe-Xaa-Xaa (SEQ ID NO: 1) and wherein in the formula of said sequence Xaa between Trp and His represents an arbitrary amino acid and the two C terminal Xaa residues either both denote Gly or the first denotes Glu and the second denotes Arg or Lys, in particular the anticancer drug is Mertansine.
